(19) United States
(12) Patent Application Publication
(10) Pub. No.: US 2012/0158109 A1
(43) Pub. Date: Jun. 21, 2012

(54) IMPLANTABLE DEVICE

(76) Inventors: Klaus BARTELS, Berlin (DE); Timo Frenzel, Berlin (DE); Stefan Knorr, Berlin (DE)

(21) Appl. No.: 13/302,466

(22) Filed: Nov. 22, 2011

Related U.S. Application Data
(60) Provisional application No. 61/424,690, filed on Dec. 20, 2010.

(51) Int. Cl. A61N 1/05

(52) U.S. Cl. 607/119

(57) ABSTRACT

An implantable medical device having at least one electrical conductor that extends longitudinally and includes a functional lead. The functional lead is connected to an electrode pole to discharge therapeutic signals or to detect diagnostic signals, wherein the functional lead or the electrode pole, or both the functional lead and the electrode pole, are constructed with a ring shape in a first longitudinal section. The electrical conductor includes at least one second electrical lead which is routed in a spiral shape in the first longitudinal section in such a manner that electromagnetic radio frequency waves which can be conducted in the first electrical lead can be coupled into the second electrical lead in the first longitudinal section.

This application claims the benefit of U.S. Provisional Patent Application No. 61/424,690 filed on 20 Dec. 2010, the specification of which is hereby incorporated herein by reference.

### BACKGROUND OF THE INVENTION

1. Field of the Invention

At least one embodiment of the invention relates to a permanently or temporarily implantable medical device having an electrical conductor that extends longitudinally.

2. Description of the Related Art

Such devices, for example electrode conductors for electrical stimulation, have the disadvantage that the electrical leads thereof can heat up in an MRI machine, because the alternating magnetic fields in an MRI machine induce electrical currents in the electrical conductor that are not insignificant. For this reason, patients with heart pace makers usually cannot be examined in an MRI using current technology, or can only be examined in a limited manner.

Implantable heart pace makers or defibrillators typically have at least one stimulation electrode lead attached to said pace maker, wherein said electrode lead has a standardized electrical connection at its proximal end, said end being provided for connection to the heart pace maker or defibrillator, and said electrode lead has one or multiple electrode poles on its distal end, said distal end being provided for locating the same in the heart. Such an electrode pole serves to release an electrical impulse, for instance to the tissue (myocardial) of the heart, or to sense electrical fields in order to be able to sense an activity such as heart activity.

For this purpose, electrode poles typically form electrically conductive surface sections of an electrode lead. Electrode poles are typically provided in the form of a ring around the electrode lead, or in the form of a point or tip electrode at the distal end of the electrode lead.

The electrode poles are connected to contacts of the electrical connection of the electrode conductor on the proximal ends thereof, in an electrically-conducting manner via one or multiple electrical leads. Consequently, one or multiple electrical leads, which electrically connect one or multiple electrode poles to one or multiple contacts, run between the contacts of the electrical connection of the electrode conductor at the proximal end thereof and the electrode poles at the distal end thereof. These electrical leads can be used to both transmit stimulation impulses to the electrode poles and also to transmit electrical signals obtained by means of the electrode poles to the proximal end of the electrode conductor. In the following description, the same are also characterized as functional leads.

Such functional leads are electrical leads which are necessary for the functions of the electrode conductor. As such, they are subject to the danger that electrical current can be induced in them by external alternating magnetic fields. This electrical current can, for instance, result in an undesirable heating of the functional leads or of the electrode poles connected to the same, or can lead to a discharge of corresponding current via the electrode poles into the surrounding tissue, thereby heating the surrounding tissue.

### BRIEF SUMMARY OF THE INVENTION

The problem addressed by at least one embodiment the invention is that of creating a medical device which solves the problem described above.

According to at least one embodiment of the invention, this problem is solved by a temporary or permanent implantable medical device having at least one electrical conductor that extends longitudinally and includes a functional lead. The functional lead is connected to an electrode pole to discharge therapeutic signals or to detect diagnostic signals, wherein the functional lead or the electrode pole, or both the functional lead and the electrode pole are designed with a ring shape in a first longitudinal section. The electrical conductor includes at least one second electrical lead which is routed in a spiral shape in the first longitudinal section in such a manner that electromagnetic radio frequency waves which can be conducted in the first electrical lead can be at least partially coupled into the second electrical lead in the first longitudinal section.

The medical device according to at least one embodiment of the invention aims to reduce undesired heating of the functional lead or of an electrode pole connected to the same, said heating being caused by electrical currents which can be induced in the functional lead of the electrical conductor by external alternating magnetic fields. In this way, at least one embodiment of the invention reduces undesired heating of bodily tissues when the device is in the implanted position, or at least partially displaces heating to other tissue regions, or even entirely avoids heating. According to at least one embodiment of the invention, this is achieved by means of a coil-shaped second electrical lead in the first longitudinal section, and by means of a coupling between the first and the second electrical lead, said coupling being designed to at least partially couple radio frequency waves which are received in the first lead into the second electrical lead.

In addition, the mechanical characteristics of at least one embodiment of the invention are improved by means of this additional lead. Such improvements include, for example, optimized resistance to bending, and rigidity with respect to mechanical influences such as crushing, flexing, tensile loading, and torsion. This decreases the risk of a so-called “subclavian crush”.

Additional features of the individual embodiments can be combined with each other to form further embodiments of the medical device in instances where those alternatives are not expressly described as exclusive to each other.

The functional lead or the electrode pole, or both the functional lead and the electrode pole are designed with a ring shape in a first longitudinal section. Both the functional lead and the electrode lead can assume the ring shape by a lead being coiled into a spiral shape. Particularly, in one embodiment, the electrode pole designed with a ring shape is formed by the functional lead in the first longitudinal section, said functional lead being uninsulated and spiral-shaped.

In preferred embodiments, the second electrical lead includes no functional lead, but rather has one or multiple additional leads.

The functional leads and the second electrical lead are preferably routed on a common, dedicated cylindrical sheath surface. The functional lead and the second electrical lead can be routed coradially in the first longitudinal section as a pair of leads which are electrically insulated with respect to each other.

In alternative embodiments, the functional lead and the second electrical lead are coiled around a solid or hollow core. The embodiment comprising coiling around a hollow core is characterized by the electrical lead having higher compressibility in the longitudinal direction.

The functional lead can be constructed outside of the first longitudinal section as either coil-shaped or as a feed cable, like a rope or cable conductor.

In the case where the functional lead is routed in a coil-shape, the electrical lead has an insulating, hollow-cylinder sheathing which has a lumen on its inside, and an inner coil of the first electrical lead is routed in the lumen. Alternatively, the feed cable can be embedded into the sheathing outside the first longitudinal section.

In one embodiment, the second electrical lead is enclosed in a lead insulation in the first longitudinal section, in order to minimize to the greatest possible extent a release of coupled energy from the second electrical lead into the surrounding bodily tissues near to the electrode pole. In other longitudinal sections, the second electrical lead can otherwise be designed as partially or entirely uninsulated.

In order to simplify the manufacturing process for this embodiment, the second electrical lead has a different diameter than the functional lead as an advantageous characteristic. This can be realized by varying wire gauges or insulation thicknesses, thereby simplifying removal of insulation from the second electrical lead in the passive area of the electrode pole.

The second electrical lead can be coiled around a sheathing of the first electrical lead in longitudinal sections which are outside the first longitudinal section. In this case, the second electrical lead is preferably partially uninsulated in an active segment—that is, a portion which is different from a functional electrode pole. However, said second lead is insulated in the non-active part.

In a further embodiment, the functional lead and the second electrical lead are embedded in the first longitudinal section into an electrically insulating sheathing of the first electrical lead.

For the purpose of increasing the coupling between the functional lead and/or the electrode pole—in the first section—and the second electrical lead, in one embodiment the second electrical lead protrudes beyond the first longitudinal section in the proximal, the distal, or both the proximal and distal directions, said second electrical lead being coiled in a spiral shape.

In preferred embodiments, the longitudinally extended electrical conductor is a temporarily or permanently implantable electrode lead designed to connect one or multiple functional electrode poles to a control device, such as for example the control device of an implantable heart pace maker or an implantable defibrillator. However, the longitudinally extended conductor as such forms a medical device in and of itself in the sense of the present description.

In addition to the embodiments described herein other alternative embodiments may include some or all of the disclosed features.

### BRIEF DESCRIPTION OF THE DRAWINGS

Additional embodiments are explained below using the figures. Shown below are:

FIG. 1 an embodiment of the invention, in the form of a heart pace maker;

FIG. 2 in partial figures, FIG. 2A showing a schematic partial cutaway view of an electrode conductor, and FIG. 2B showing a cross-sectional view of the electrode conductor from FIG. 2A;

FIG. 3 in partial figures, FIG. 3A showing a schematic side view of an electrode conductor, and FIG. 3B showing a cross-sectional view of the electrode conductor from FIG. 3A;

FIG. 4 in partial figures, FIG. 4A showing a schematic side view of an electrode conductor, and FIG. 4B showing a cross-sectional view of the electrode conductor from FIG. 4A;

FIG. 5 in partial figures, FIG. 5A showing a schematic side view of an electrode conductor, and FIG. 5B showing a cross-sectional view of the electrode conductor from FIG. 5A;

FIG. 6 in partial figures, FIG. 6A showing a schematic cutaway view of an electrode conductor, and FIG. 6B showing a cross-sectional view of the electrode conductor from FIG. 6A;

FIG. 7 in partial figures, FIG. 7A showing a schematic partial side view of an electrode conductor, and FIG. 7B showing a cross-sectional view of the electrode conductor from FIG. 7A;

### DETAILED DESCRIPTION OF THE INVENTION

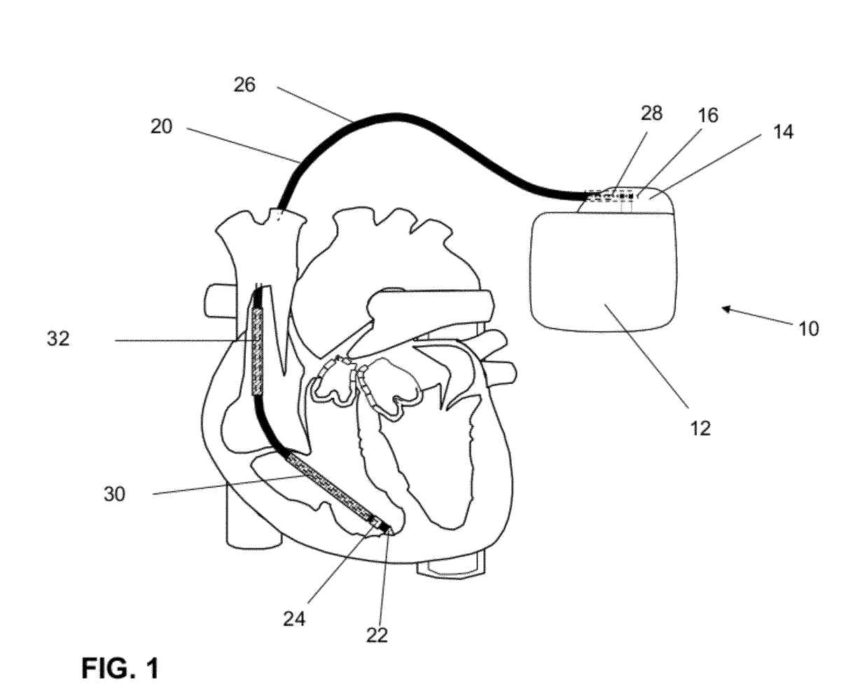

FIG. 1 shows, as an example of implantable medical devices, an implantable heart stimulator 10 and an implantable electrode conductor 20 connected to the same.

The implantable heart stimulator 10 can be a heart pace maker or a cardioverter/ defibrillator (ICD). In the illustrated embodiment, the heart stimulator 10 is a ventricular heart pace maker and defibrillator. Other known heart stimulators are two-chamber heart pace makers designed to stimulate the right atrium and the right ventricle, or biventricular heart pace makers which can stimulate the left ventricle in addition to the right ventricle.

Such stimulators typically have a housing 12, which usually is made of metal, and is consequently electrically conducting and can serve as a large-surface area electrode pole. Typically, a connection housing 14 is attached to the outer side of the housing, and is also called a ‘header’. Such a header typically has contact connectors as receptacles for plug contacts. The contact connectors have electrical contacts 16 which are connected via appropriate leads to the electronics arranged inside the housing 12 of the heart stimulator.

The electrode conductor 20 likewise constitutes an implantable medical device in the sense of this invention. Electrode poles, in the form of a point or tip electrode 22 as well as one ring electrode 24 arranged near said electrode poles, are arranged at the distal end of the electrode conductor 20 in a conventional manner. The electrode poles 22 and 24 are designed in such a manner that they serve to sense electrical potentials of the (myocardial) heart tissue, or they serve to discharge electrical signals, for instance to release stimulation impulses to the heart tissue surrounding the electrodes, according to the function of the heart stimulator to which the electrode conductor 20 is attached. FIG. 1 shows how the electrode poles, that is, the tip electrode 22 and the ring electrode 24, and in certain cases the electrode conductor 20, are located in the apex of a right ventricle of a heart.

Both the tip electrode 22 and the ring electrode 24 are electrically connected to a plug contact 28 at the proximal end of the electrode conductor 20 via at least one electrical lead 26 each. The plug contact 28 has electrical contacts which correspond to the electrical contacts 16 of the contact connector in the connection housing 14 of the implantable heart stimulator.

As described in greater detail below, the electrical leads 26 in the electrode conductor 20 can be constructed in different longitudinal sections as primarily extended feed cables or as helix-shaped coiled leads. Such leads, which connect the functional electrode poles with electrical contacts of the plug contact on the proximal end of the electrode conductor 20 in an electrically conducting manner, are also characterized in the scope of this description as functional leads because they transmit therapeutic electric signals from the plug contact to one or both electrode poles, or they convey sensed electrical potentials to the plug contact, said potentials representing signals from one or both electrode poles. Consequently, said leads serve to fulfill the elementary function of the medical device.

The functional electrical leads 26, which connect the electrode pole 22 and/or 24 to the electrical contacts of the plug 28 of the electrode conductor 20, are enclosed over the majority of their length by an insulating sheathing, such that electrical contact to the tissue of the heart occurs in a targeted manner at the electrode poles.

In addition to the electrode poles 22 and 24, which typically serve to stimulate the heart tissue (in this case, ventricular tissue), the electrode conductor 20 also has two additional large surface area electrode poles 30 and 32, which function as defibrillation electrodes and are formed by at least one uninsulated helix-shaped coiled wire.

An embodiment of the invention in the form of a right ventricular heart pace maker and defibrillator in utilized to explain operation. However, in principle, an ablation electrode lead could also be adduced as an example of a medical device in the sense of at least one embodiment of the invention, said ablation electrode lead likewise projecting into the heart of a patient and being controlled by a device outside the patient's body and, for that purpose, connected to the same. Furthermore, such electrode leads can also function in other applications, upon technical adjustment for the special requirements of other specific uses, to stimulate tissue or relay signals to/from nerves, the brain, and other organs, or as feeds from implantable sensors.

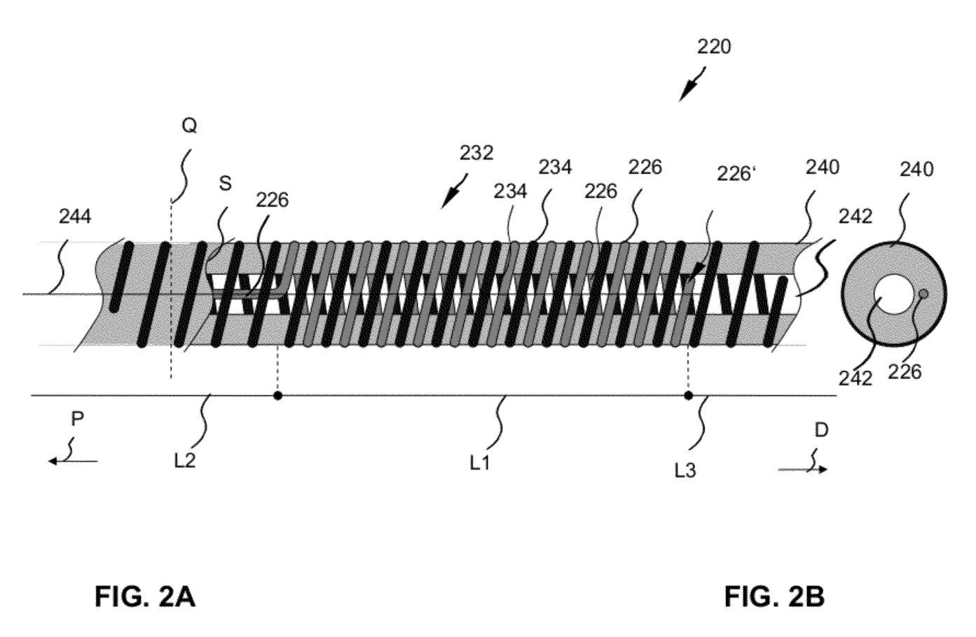

FIG. 2 shows, in its partial figures, FIG. 2A: a schematic partial cutaway view of an electrode conductor, and FIG. 2B: a cross-sectional view of the electrode conductor from FIG. 2A. The electrode conductor 220 illustrated in FIG. 2A is shown only in a sub-section of the longitudinal extension of the same. This sub-section corresponds in the present embodiment to a part of the electrode conductor 20 arranged in the right atrium of the heart in FIG. 1, said part of the electrode containing the electrode pole 32. The sub-section can, however, likewise illustrate the section having the electrode pole 30, lying further towards the distal end. For the purposes of the present description, the reader can generally assume that the sub-section corresponding to electrode pole 32 is illustrated, without restriction to the content of the description.

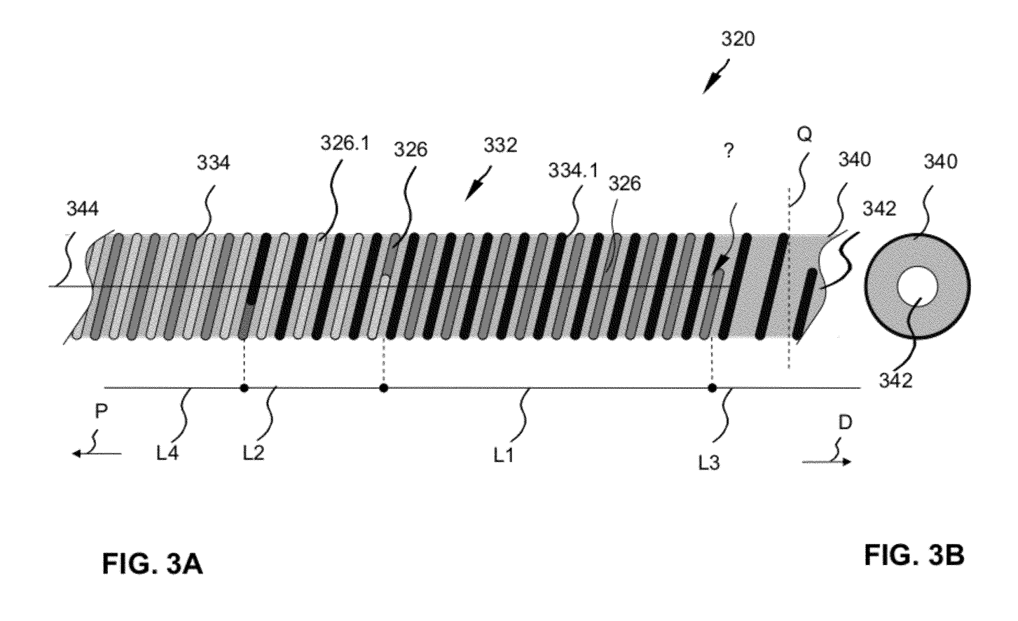

For the purpose of clarifying this context, similar reference numbers are used for the electrode lead and for said electrode pole in the present illustration as in FIG. 1. However, an “Arabic numeral 2” is prefixed to the reference number given in FIG. 1. Because the other embodiment examples in FIGS. 3 to 7 also refer to the description below, this concept will also be used there. However, the same functional elements will be prefixed to each of the reference numbers used in FIG. 1, said reference numbers corresponding to the figure numbering, in order to identify different variants of the functional elements and their design.

FIG. 2A now shows a partial longitudinal side view of the electrode conductor 220. In the cutaway view of FIG. 2A, the electrode conductor 220 is illustrated beyond a cross section S in a distal direction D, having a longitudinally oriented sheathing. This serves merely to clarify the construction of the electrode conductor 220, which in fact has a continuous and closed sheathing 240, as is visible on this side of the cross section in a proximal direction P.

A functional lead 226 is routed as a cable feed in the sheathing 240, from the proximal end (not illustrated here) of the electrode conductor 220 to the beginning of a first longitudinal section L1, which contains the electrode pole 232. The routing in the sheathing 240 can be seen in the cross-sectional view given in FIG. 2B. The cross-section plane Q is identified in FIG. 2A by a dashed line.

In the first longitudinal section L1, the electrode pole 232 is constructed with a substantially spiral shape, in which the functional lead 226 is routed with a spiral shape, and no longer as a feed cable. The functional lead 226 is uninsulated in this first longitudinal section L1, and coiled around the cylindrical sheath surface of the sheathing 240. The functional lead 226 is, on the other hand, coiled inside the sheathing 240 as it connects to the first longitudinal section L1, which is not illustrated here in detail. Also, the functional lead 226 ends at position 226′ in FIG. 2A, which isn't strictly necessary. The functional lead 226 can also continue further.

The functional lead 226 is coiled around a hollow core in the electrode pole 232 area. In this longitudinal section, and in sections which connect thereto both proximally and distally, an additional lead 234 is coiled around the sheathing 240 in a spiral manner. In the electrode pole 232 area, the functional lead 226 and the additional lead 234 are coiled around a hollow core coradially. This means that the functional lead 226 and the additional lead 234 are coiled in parallel next to each other around the sheathing 240 in a cylindrical sheath surface formed by the outer surface of the sheathing 240. Instead of one additional lead 234, two or more additional leads can also be used.

The additional lead 234 and the functional lead 226 are electrically insulated from each other. This can, for example, be achieved even without insulation of both leads, by virtue of the clearance space in the longitudinal direction between the two leads, and by their coiling on the insulating material of the sheathing 240. For the sheathing 240, materials including silicon and any other suitable material can be used, the same possessing the proper mechanical, electrical, and biological characteristics for the relevant application. Alternatively, the functional lead 226 and the additional lead 234 are insulated with respect to each other by a lead insulation of the additional lead 234.

The additional lead projects beyond the first longitudinal section L1, in both the proximal direction P into a second longitudinal section L2, and in the distal direction D into a third longitudinal section L3.

For purposes of simplicity, the present illustration does not include other details which are not immediately relevant, such as additional possible functional leads which could be routed inside a lumen 242 of the electrode lead 220. The sheathing 240 has the shape of a flexible hollow cylinder, the axis 244 of which is included in the illustration in FIG. 2A.

The structure shown in FIG. 2A and 2B enables an electromagnetic coupling between the functional lead 226 and the additional lead 234. This coupling functions to couple particularly electromagnetic radio frequency waves into the additional lead from the functional electromagnetic wave lead 226, but primarily also electromagnetic waves with frequencies which are significantly higher than the frequencies which are conventionally used for therapeutic purposes and for diagnostic purposes. Because high frequency electromagnetic signals can be damaging to the functional lead in therapeutic applications, as described above, by utilizing the electrode lead 220 it is possible to avoid such damage and ensure uncompromised function of the electrode lead, even in the presence of high-frequency alternating electromagnetic fields. In addition to an inductive coupling, a capacitative coupling exists between the functional lead 226 and the additional lead 234. This can be modified by means of the selection of insulating material and the clearance gap between the two leads. The larger the clearance gap is between both leads in the longitudinal direction of the electrode conductor 220, the smaller the coupling is.

The amplitude of the signal which is coupled into the additional lead can be modified by a longitudinal extension of the first longitudinal section L1. The larger the length of the coupling is, the larger the amplitude of the coupled signal is until reaching saturation.

FIG. 3 shows, in its partial figures, FIG. 3A: showing a schematic side view of an electrode conductor, and FIG. 3B: showing a cross-sectional view of the electrode conductor from FIG. 3A. The electrode conductor in FIGS. 3A and 3B is similar in many features to the electrode conductor 220 in FIGS. 2A and 2B. The following description therefore concentrates only on the differences between the two embodiments.

Unlike in the example illustrated by FIGS. 2A and 2B, in the present embodiment example the functional lead 326 is also coiled in a spiral around the sheathing 340 beyond the first longitudinal section L1, and the section even extends in the proximal direction P, with a spiral-shaped routing, into longitudinal sections L2 and L4. In this case, the functional lead is combined throughout longitudinal sections L1, L2, and L4 in a coradial manner with the additional lead 334 and coiled around a hollow core.

In the present embodiment example, in which the additional lead could also be characterized as a drain coil, the additional lead is uninsulated in one or more longitudinal sections. As an example, the additional lead 334 is uninsulated in longitudinal section L4, while it is insulated in longitudinal section L2, where the same continues, as well as in the connecting longitudinal sections L1 and L3. Alternatively, the lead insulation in the previously mentioned three longitudinal sections can also be continued if the additional lead is sufficiently insulated from the functional lead 326 by the insulating material of the sheathing 340.

The functional lead 326 is, as in the previous embodiment example, uninsulated in the first longitudinal section L1, in order to enable its therapeutic or diagnostic function. The electrode lead 332 of the present embodiment example enables an increased inductive and capacitative coupling between the functional lead 326 and the additional lead 334 due to its different features. Moreover, it enables the release of electromagnetic energy outside of the first longitudinal section L1 of the functional electrode pole 332, particularly in the longitudinal area L4, where the additional lead 334 is not insulated with respect to the bodily tissues.

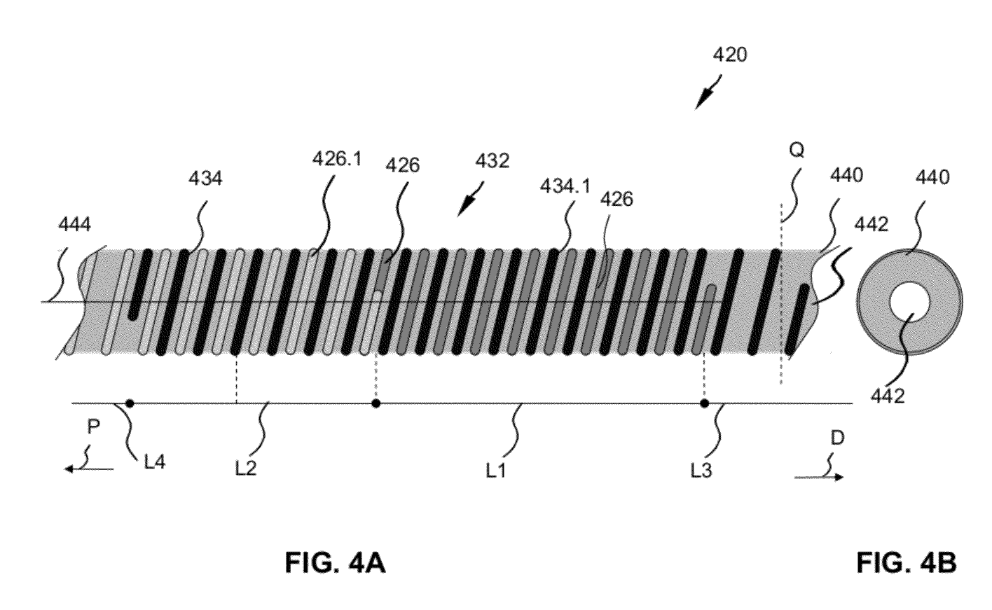

FIG. 4 shows, in its partial figures, FIG. 4A: showing a schematic side view of an electrode conductor, and FIG. 4B: showing a cross-sectional view of the electrode conductor from FIG. 4A. The electrode conductor 420 of the previous embodiment example is similar to the electrode conductor in FIG. 3. It differs therefrom only in that the additional lead 434 is insulated in all longitudinal sections in which it is routed in a coradial manner with the functional lead 426. Moreover, in the present embodiment example, the additional lead 434 extends, coiled around the sheathing 440, along the electrode conductor 420 in the proximal direction only until just behind the start of the fourth longitudinal section L4. As such, it extends longer longitudinally than the functional electrode pole 432, but runs inside the sheathing 440 when outside of the illustrated longitudinal section. This design reduces the coupling between the functional lead 426 and the additional lead 434, and limits the couple to the electrode pole 432 and its immediate surroundings.

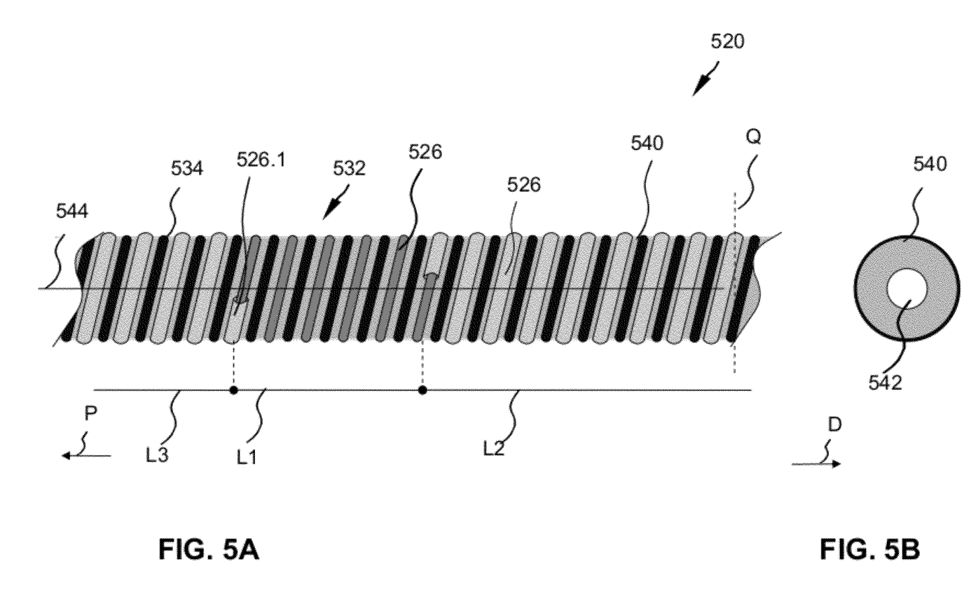

FIG. 5 shows, in its partial figures, FIG. 5A: showing a schematic side view of an electrode conductor, and FIG. 5B: showing a cross-sectional view of the electrode conductor from FIG. 5A. The electrode conductor 520 of the present embodiment example has a functional lead 526 which is insulated in longitudinal sections which lie outside of the electrode pole 532. The additional lead 534 is continuously insulated. In the present embodiment, the functional lead 526 and the additional lead 534 have different thicknesses, and as such have different wire diameter/gauge. Moreover, as an alternative, or additionally, the insulation of one of the two leads 526 and 534 can have a different diameter/gauge than the insulation of the other lead. In the present example, the functional lead 526 has a thicker insulation that the additional lead 534, and additionally has a higher wire diameter, regardless of the insulation surrounding the same. The outer diameter of the insulated functional lead in the first longitudinal area L1 of the functional electrode 532 is similar to the additional lead 534 with respect to outer diameter.

This embodiment has the advantage that the functional electrode pole 532 can be easily manufactured without damaging the insulation of the additional lead 534, by insulating the functional lead.

In other longitudinal sections which are not illustrated here, only the additional lead 534 is uninsulated, and the functional lead 526 is insulated. In this way, sections of the additional lead 534 can be in direct contact with the tissue, in order to enable heat conductance via a passive electrode area which is not used for therapeutic or diagnostic purposes. The embodiment enables a simplified selective insulation during manufacturing of the electrode lead, for the production of either a functional electrode pole or a passive electrode area.

The embodiment examples in FIGS. 2 to 5 have functional and additional leads which are coiled around a hollow core. The advantage of this hollow core coiling is its especially high compressibility.

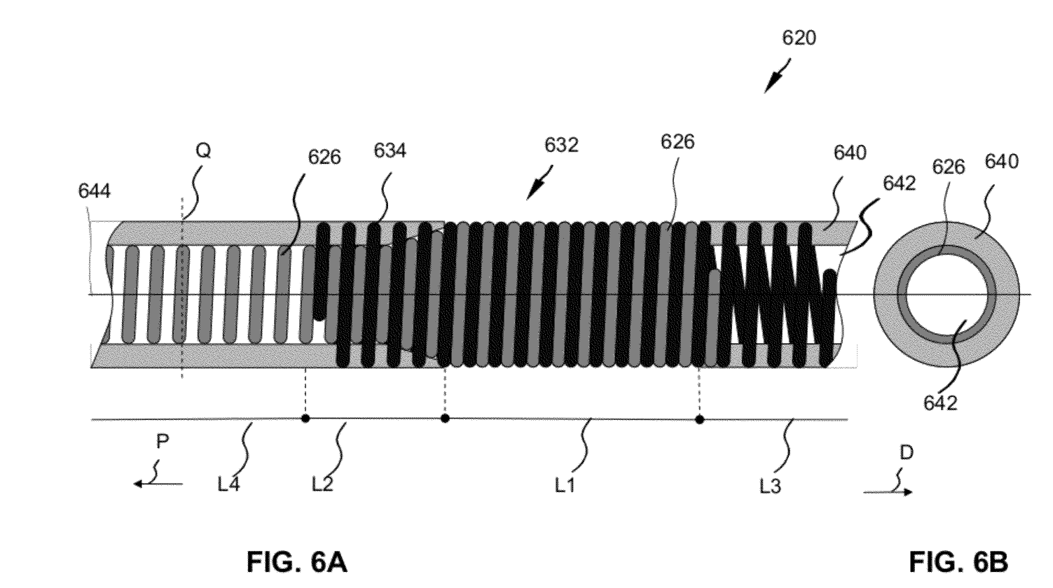

FIG. 6 shows, in its partial figures, FIG. 6A: showing a schematic cutaway view of an electrode conductor, and FIG. 6B: showing a cross-sectional view of the electrode conductor from FIG. 6A. Unlike in the previously illustrated and previously explained embodiment examples, the electrode conductor 620 has a break in the sheathing 620 in the area of the electrode pole 632. In the corresponding longitudinal section L1, the functional lead 626 and the additional lead 635 are coiled in a spiral coradially around a solid core. The functional lead 626 and the additional lead 634 are insulated with respect to each other. The additional lead 634 projects beyond the longitudinal section L1 of the electrode pole 632, the same being designed as a ring electrode, proximally into longitudinal section L2 and distally into longitudinal section L3. In the present embodiment, the functional lead 626 is routed in a spiral shape continuously, and extends into longitudinal section L4, that is, from a proximal electrode connection to the area of the electrode pole 632, through the lumen 642 of the sheathing 640. This embodiment example has a particularly strong inductive and capacitative coupling between the functional lead 626 and the additional lead 634, particularly in longitudinal section L1 of the electrode pole 632.

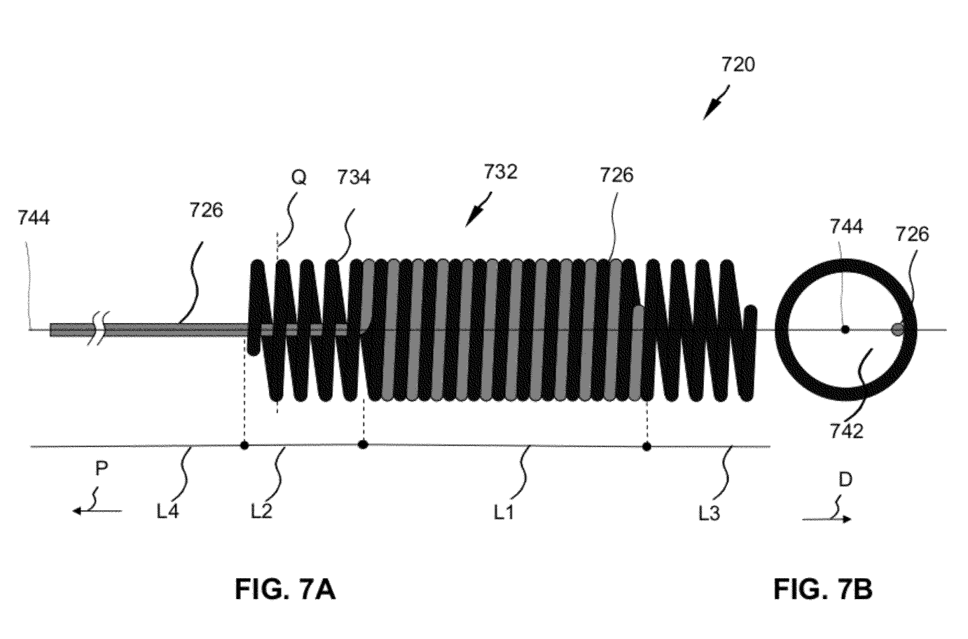

FIG. 7 shows, in its partial figures, FIG. 7A: a schematic partial side view of an electrode conductor, and FIG. 7B: a cross-sectional view of the electrode conductor from FIG. 7A. However, in both cases a sheathing is present, which is not illustrated here. The embodiment examples in FIG. 7A and FIG. 7B only differ from those in FIG. 6 in that the functional lead 726 is routed as a feed cable up to longitudinal section L1 of the electrode pole 732. The functional lead is routed in the lumen of the electrode lead 720 as a feed cable in these longitudinal sections L2 and L4, and namely not around a central axis of the hollow cylinder formed by the electrode pole 732.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. An implantable medical device comprising an electrical conductor that extends longitudinally and which comprises
an electrode pole;
a functional lead which is connected to the electrode pole to discharge or detect therapeutic signals wherein the functional lead or the electrode pole, or both the functional lead and the electrode pole, are configured in a ring shape in a first longitudinal section; and,
a second electrical lead which is routed spirally in the first longitudinal section in such a manner that electromagnetic radio frequency waves which are conducted in the functional lead can be at least partially coupled into the second electrical lead in the first longitudinal section.

2. The medical device according to claim 1, wherein the electrode pole in said ring shape comprises the functional lead in the first longitudinal section, said functional lead being uninsulated and spiral shaped.

3. The medical device according to claim 1, wherein the second electrical lead includes no functional lead, but rather has one or multiple additional leads.

4. The medical device according to claim 1, wherein the functional lead and the second electrical lead are routed on a common, dedicated cylindrical sheath surface in a spiral manner.

5. The medical device according to claim 4, wherein the functional lead and the second electrical lead are routed coradially in the first longitudinal section as a pair of leads that are electrically insulated with respect to each other.

6. The medical device according to claim 2, wherein the functional lead and the second electrical lead are coiled on a solid core in the first longitudinal section.

7. The medical device according to claim 2, wherein the functional lead and the second electrical lead are coiled on a hollow core in the first longitudinal section.

8. The medical device according to claims 2, wherein the second electrical lead is surrounded by lead insulation in the first longitudinal section.

9. The medical device according to claim 2, wherein the second electrical lead has a different diameter than the functional lead.

10. The medical device according to claim 2, wherein the functional lead comprises an electrically insulating sheathing and wherein the functional lead and the second electrical lead are embedded in the electrically insulating sheathing of the functional lead in the first longitudinal section.

11. The medical device according to claim 2, wherein the second electrical lead protrudes beyond the first longitudinal section in a proximal, a distal, or both the proximal and distal directions, wherein said second electrical lead is coiled in a spiral manner.

12. The medical device according to claim 11, wherein the functional lead comprises a sheathing and wherein the second electrical lead is coiled around the sheathing of the functional lead in longitudinal sections which are outside the first longitudinal section.

13. The medical device according to claim 1, wherein the functional lead is configured as a feed cable outside the first longitudinal section.

14. The medical device according to claim 1, having a hollow cylindrical sheathing which has a lumen inside, wherein an inner spiral of the functional lead is routed inside said lumen.

15. The medical device according to claim 13, further comprising sheathing wherein the feed cable is embedded in the sheathing outside of the first longitudinal section.
